(19) United States
(12) Patent Application Publication
(10) Pub. No.: US 2025/0338832 A1
(43) Pub. Date: Nov. 6, 2025

(54) METHOD FOR TETRAPLOID INDUCTION IN FISH

(71) Applicants: University of Idaho, Moscow, ID (US); Evergreen Aquatics, La Center, WA (US)

(72) Inventors: Luke Oliver, Moscow, ID (US); Kenneth D. Cain, Port Orchard, WA (US); Colby Johnson, La Center, WA (US)

(73) Assignees: University of Idaho, Moscow, ID (US); Evergreen Aquatics, La Center, WA (US)

(21) Appl. No.: 19/196,429

(22) Filed: May 1, 2025

Related U.S. Application Data
(60) Provisional application No. 63/642,253, filed on May 3, 2024.

(51) Int. Cl. A01K 67/027

(52) U.S. Cl. CPC A01K 67/027; A01K 2207/35; A01K 2227/40

(57) ABSTRACT

Disclosed herein are aspects of a method for inducing tetraploid production in fish eggs, to produce a population of tetraploid fish. The method may comprise selecting fish eggs at a desired first cleavage interval and exposing the eggs to a pressure shock to induce tetraploidy. Also disclosed herein are methods of using tetraploid fish to produce a sterile population of fish, for example for aquaculture or to reduce a population of an invasive species in a water system.

### CROSS REFERENCE TO RELATED APPLICATION

This application claims the benefit of U.S. Provisional Application No. 63/642,253, filed May 3, 2024, which is incorporated herein by reference in its entirety.

### ACKNOWLEDGMENT OF GOVERNMENT SUPPORT

This invention was made with government support under Grant No. 2023-33530-39342 awarded by U.S. Department of Agriculture National Institute of Food and Agriculture (USDA-NIFA). The government has certain rights in the invention.

### FIELD

Disclosed herein are aspects of a method to induce tetraploid production in fish.

### BACKGROUND

The manipulation of chromosome sets is often used in aquaculture to induce sterility, as sterile stocks eliminate invasive potential, deny unauthorized reproduction of intellectual property, and may be required by law in some situations. Sexually reproducing species with odd chromosome sets are generally sterile and those with even chromosome sets are usually fertile. Fish ovulate eggs in a non-reduced state, fertilization triggers the release of the second polar body and results in a diploid (2N) zygote. When applied at the crucial period with adequate intensity and duration, physical shocks (usually thermal or hydrostatic) prevent the expulsion of the second polar body during meiosis in fertilized fish eggs and this results in a triploid (3N) zygote.

Burbot (Lota lota), the only freshwater species of cod-like fish (gadiformes), has great potential for commercial cool or cold-water aquaculture. Captive husbandry methods are well understood, the species performs well on commercial salmonid or carnivorous marine fish diets, and the products from this fish (meat, liver, roe, and skin) are well accepted by consumers. Furthermore, this species is refractory to many salmonid pathogens, and recently developed immune assays have shown immunological protection in response to vaccination or pathogen exposure. Finally, triploid burbot show promise as adults are sterile (sperm is aneuploid and eggs are nonfunctional) and may exhibit greater growth rate relative to adult diploids.

However, the induction rate of shock-induced triploids is often less than 100%, meaning that some resulting fish may be fertile. And the shocks used to induce triploidy contribute to pre-hatch mortality.

### SUMMARY

Disclosed herein are aspects of a method for inducing tetraploid production in fish eggs and larvae, such as burbot eggs and larvae. The method comprises selecting fish eggs at between about 60% to about 75% of the first cellular cleavage interval (FCI), such as from about 65% to about 75% FCI, and exposing the eggs to a pressure shock comprising a pressure from about 7000 psi to about 9000 psi that is applied for from about 1° C. minute to about 20° C. minutes. In some aspects, the pressure may be from about 7,250 psi to about 8,500 psi, or about 7,500 psi. And in other aspects, exposing the eggs to the pressure shock comprises exposing the eggs to the pressure for from about 1° C. minute to about 10° C. minutes, such as from about 7° C. minute to about 10° C. minutes, or about 5 minutes.

In some aspects, the method is a method for inducing from about 50% to 100% tetraploid production, such as from about 60% to 100% or from about 65% to 100% tetraploid production in the eggs and larvae.

In some aspects, the fish eggs are eggs from a species of fish, such as a cold-water species of fish. The cold-water species may be a freshwater species or a marine species.

In some aspects, the fish eggs have an average size of from greater than zero to about 5 mm, such as from about 0.5 mm to about 3 mm or from about 0.7 mm to about 2 mm. And in other aspects, the fish are selected from carp, walleye, yellow perch, burbot, Yellowtail Kingfish, an amberjack species (genus Seriola), sablefish (black cod), red drum, cobia, flounders, Atlantic cod, pacific cod, sea bass species, mahi mahi, tilapia, largemouth bass, or catfish.

In any aspects, the survival rate of larvae from the eggs that are exposed to the pressure shock at about 60% to about 75% FCI is about 10% or greater compared to the survival rate of larvae from fish eggs that have not been exposed to the pressure shock, such as about 12% or greater. And in some aspects, the survival rate is about 15% or greater, and maybe about 20% or greater, about 25% or greater or about 30% or greater. In certain aspects, the FCI is from about 60% to less than about 75% and the survival rate is about 15% or greater, such as about 20% or greater. And in other particular aspects, the FCI is from about 65% to about 70% and the survival rate is about 30% or greater.

In a particular aspect, the method is a method of inducing from about 85% to 100% tetraploid production, comprising selecting fish eggs at between about 65% to about 75% FCI, and exposing the eggs to a pressure shock comprising a pressure of from about 7,250 psi to about 7,750 psi, such as about 7,500 psi for from about 3° C. minutes to about 7° C. minutes, such as about 5° C. minutes. And the method may further comprise allowing the eggs to hatch to produce larvae having from about 85% to 100% tetraploidy.

Also disclosed herein are aspects of a method for producing a sterile population of fish, comprising introducing a population of tetraploid fish of a selected species to a population of diploid fish of the selected species as the tetraploid fish, and allowing the tetraploid fish to breed with the diploid fish, thereby producing a population of sterile triploid fish of the selected species.

Further disclosed are aspects of a method of reducing a population of an invasive species of fish in a water system, the method comprising introducing to the water system a plurality of tetraploid fish of the invasive species.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a digital image of burbot eggs, about 1 mm in diameter, at about 7 hours post fertilization.

FIG. 2 is a digital image of burbot eggs, about 1 mm in diameter, at about 1 hour prior to first cleavage, with some early cleavage cleft being apparent in some eggs.

FIG. 3 is a digital image of burbot eggs, about 1 mm in diameter, illustrating first cleavage.

FIG. 4 is a graph of count of events versus nuclear acid values in arbitrary units of fluorescence intensity (PE-A) from flow cytometry analysis of nucleic acid from burbot (Lota lota), illustrating the results of 50,000 events that were recorded from diploid burbot blood.

FIG. 5 is a graph of count of events versus nuclear acid values in arbitrary units of fluorescence intensity (PE-A) from flow cytometry analysis of nucleic acid from rainbow trout (Oncorhynchus mykiss), illustrating the results of 50,000 events that were recorded from diploid rainbow trout blood.

FIG. 6 is a graph of count of events versus nuclear acid values in arbitrary units of fluorescence intensity (PE-A) from flow cytometry analysis of nucleic acid from burbot (Lota lota) and rainbow trout (Oncorhynchus mykiss), illustrating the results of 50,000 events that were recorded from diploid burbot blood and diploid larval sample and rainbow trout blood.

FIG. 7 is a graph of count of events versus nuclear acid values in arbitrary units of fluorescence intensity (PE-A) from flow cytometry analysis of nucleic acid from burbot (Lota lota) and rainbow trout (Oncorhynchus mykiss), illustrating the results of 50,000 events that were recorded from diploid burbot blood, tetraploid burbot larva, rainbow trout blood.

FIG. 8 is a graph of percent tetraploid induction per replicate versus shock application relative to percent of first cleavage interval, illustrating tetraploid induction, with percent per replicate (squares) and average for each treatment (diamonds) including standard error. The alternating color scheme, filled black data points or unfilled white data points, are included to make visual assessment by treatment easier.

### DETAILED DESCRIPTION

### I. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Unless context indicated otherwise, “about” refers to plus or minus 5% of a reference value. For example, “about” 100 refers to 95 to 105. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood to one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

### II. Overview

Triploids can be produced by crossing a diploid with a tetraploid (4N), since gametes are relatively haploid (half the respective N) the resulting progeny is triploid. Triploids derived from tetraploid x diploid crosses (autotriploid) may have several advantages relative to shock-induced triploids. Offspring from tetraploid x diploid crosses are all triploid, while the induction rate of shock-induced triploids is often less than 100%. The shocks used to induce triploidy also contribute to pre-hatch mortality. However, autotriploids are not exposed to shocks and thus experience less pre-hatch trauma. Performance of autotriploid fish has been observed to be comparable to diploids and slightly better than shocked-induced triploids.

Tetraploids have been produced in several fish species. However, tetraploid yield and survival rates were low. Because of this it has been difficult to produce viable mature and fertile tetraploids for important species. For example, in Malison et al. (“Manipulation of ploidy in yellow perch (Perca flavescens) by heat shock, hydrostatic pressure shock, and spermatozoa inactivation” Aquaculture, (1993) 110(3-4), pp. 229-242) pressure shocks of 9000 psi were administered to fertilized yellow perch eggs to try to induce tetraploidy in yellow perch. However, in their method, the eggs were at 11° C. and the pressure was applied for 8, 16 or 24 minutes, which equates to 88° C. minutes, 176° C. minutes and 264° C. minutes (11° C.×8 minutes, 16 minutes, or 24 minutes, respectively). These values are substantially greater than the degree minutes disclosed herein, which are from about 1° C. minute to about 20° C. minutes. Additionally, to achieve survival rates of 10% or greater and/or tetraploid induction of over 50%, Malison et al. had to perform the pressure shock treatment at a TI of 192 minutes, which equates to about 90% FCI. Again, these conditions are substantially different and harsher to the milder conditions disclosed herein.

### III. Method of Inducing Tetraploid Production

Disclosed herein is a method of inducing tetraploid production in fish larvae. In some aspects, the method comprises selecting fish eggs having a desired first cleavage interval (FCI) and exposing the eggs to a pressure shock to induce tetraploidy. The eggs are then allowed to hatch to produce tetraploid fish larvae. In some aspects, tetraploid production in the larvae is from about 50% to 100%, such as about 60% to 100%, about 65% to 100%, about 70% to 100%, about 75% to 100%, about 80% to 100%, about 85% to 100%, about 90% to 100%, or about 95% to 100%.

FCI may be determined by fertilizing a small fraction of eggs from an individual female, monitor for the formation of the first zygote cleavage, then fertilize the remaining eggs and apply the pressure shock based on the calculated FCI. In some aspects, the FCI used for the disclosed method is from about 50% to about 85%, such as from about 55% to about 80%, from about 60% to about 75%, or from about 65% to about 75%.

In some aspects, the pressure shock comprises exposing the eggs to a pressure of from about 5,000 psi to about 10,000 psi, such as from about 6,000 psi to about 9,000 psi, from about 7,000 psi to about 9,000 psi, from about 7,000 psi to about 8,500 psi, from about 7,250 psi to about 8,500 psi, from about 7,250 psi to about 8,250 psi, or from about 7,250 psi to about 8,000 psi. In certain aspects, the pressure is about 7,500 psi.

In some aspects, the pressure shock also comprises exposing the eggs to the pressure for a time period of from about 1° C. minute to about 20° C. minutes, or longer, such as from about 1° C. minutes to about 15° C. minutes, from about 1° C. minute to about 10° C. minutes, from about 1° C. minutes to about 5° C. minutes, from about 2.5° C. minutes to about 10° C. minutes, or from about 5° C. minutes to about 10° C. minutes. As used herein a degree minute (° C. minute) is time multiplied by temperature. In some aspects, the eggs were at a temperature of from about 1° C. to about 5° C. before the pressure shock was applied, such as from about 1° C. to about 3° C., or about 2° C.

Fish eggs suitable for use in the disclosed method include any fish eggs that can be investigated to determine cleavage. The species of fish may be a cold-water species and may be a marine species or a freshwater species of fish. In some aspects, the fish eggs have an average size of from greater than zero to about 5 mm, such as from about 0.5 mm to about 3 mm or from about 0.7 mm to about 2 mm. In certain aspects, the fish eggs are about 1 mm in diameter. The average size of the fish eggs is determined by measuring the longest dimension of the fish eggs.

In certain aspects, the fish eggs are eggs from fish selected from freshwater species, such as carp, walleye, yellow perch, or burbot, and/or seawater species, such as Yellowtail Kingfish and amberjack species (genus Seriola), sablefish (black cod), red drum, cobia, flounders, Atlantic and pacific cod, sea bass species, mahi mahi, tilapia, large mouth bass, or catfish. And in a particular aspect, the fish eggs are burbot eggs.

In some aspects, the survival rate for the larvae from the eggs that are exposed to the pressure shock is about 10% or greater than the survival rate of eggs that are not exposed to the pressure rate. And in certain aspects, the survival rate is about 15% or greater, and maybe about 20% or greater, about 25% or greater, or about 30% or greater.

After exposure to the pressure shock, the eggs are returned to an incubator and allowed to hatch under standard conditions that are typically used for production of the particular fish species, such as burbot. After hatching a sample of the surviving larvae are analyzed to determine tetraploid induction, for example, as described in Example 5 herein.

### IV. Applications

The disclosed method may be used to produce a population of tetraploid fish. Tetraploid fish are useful for several applications including, but not limited to, producing a sterile population of fish, and combatting an invasive species in a water system. By way of an example, a cost estimate in 2024 for eradicating invasive burbot from the Big Sandy River Basin in Wyoming was over $1.8 million. This cost includes chemical treatments of the river and reservoir and protection for the native fish during the treatment process. While this is an estimated cost for removing an invasive species from just one river, it clearly demonstrates the potential value of the disclosed technology, which may help reducing or removing the need for chemical treatments and thereby reducing the associated costs for protecting the native fish.

Typically, tetraploid fish will breed with other tetraploid fish to produce tetraploid offspring. In some aspects, a first generation of tetraploid fish, such as fish grown from eggs that are exposed to the pressure shock disclosed herein, are less robust than subsequent generations that are offspring of the first generation. Accordingly, for some applications, the tetraploid fish produced by the method disclosed herein are allowed to breed to produce at least a second generation of tetraploid fish before being used for an end use application.

In some aspects, tetraploid fish, such as fish produced by the disclosed method, are allowed to breed with a population of diploid fish to produce a population of triploid fish, which are typically sterile. A sterile population of fish is useful, for example, in aquaculture. In some areas, a sterile population of fish for aquaculture is required or desired, particularly in areas where there is a risk of aquaculture fish escaping into local waterways, for example, as a result of flooding.

In other aspects, in a water system that is infested with an invasive species of fish, tetraploid fish of the same species may be introduced. By introducing even a small percentage of the tetraploid fish, compared to the population of the invasive species, the population of the invasive species can be reduced over time. Again, the tetraploid fish breed with the invasive species diploid population thereby producing triploid offspring. Because these triploid offspring are sterile, the population of the invasive species will be reduced over time.

### V. Exemplary Aspects

The following numbered paragraphs illustrate exemplary aspects of the disclosed technology.

Paragraph 1. A method, comprising:
selecting fish eggs at between about 65% to about 75% FCI;
exposing the eggs to a pressure shock comprising a pressure from about 7000 psi to about 9000 psi for from about 1° C. minutes to about 20° C. minutes.

Paragraph 2. The method of paragraph 1, wherein the method is a method of inducing from about 50% to 100% tetraploid production.

Paragraph 3. The method of paragraph 1 or paragraph 2, wherein the method is a method of inducing from about 60% to 100% tetraploid production.

Paragraph 4. The method of any one of paragraphs 1-3, wherein the fish eggs are from a cold-water species of fish.

Paragraph 5. The method of paragraph 4, wherein the cold-water species of fish is a freshwater species.

Paragraph 6. The method of any one of paragraphs 1-5, wherein the fish eggs have an average size of from greater than zero to about 5 mm.

Paragraph 7. The method of paragraph 6, wherein the average size of the fish eggs is from about 0.5 mm to about 3 mm.

Paragraph 8. The method of paragraph 6, wherein the average size of the fish eggs is from about 0.7 mm to about 2 mm.

Paragraph 9. The method of any one of paragraphs 1-8, wherein the fish eggs are eggs from fish selected from carp, walleye, yellow perch, burbot, Yellowtail Kingfish and amberjack species (genus Seriola), sablefish (black cod), red drum, cobia, flounders, Atlantic cod, pacific cod, sea bass species, mahi mahi, tilapia, large mouth bass, or catfish.

Paragraph 10. The method of any one of paragraphs 1-9, wherein the pressure is from about 7,250 psi to about 8,500 psi.

Paragraph 11. The method of any one of paragraphs 1-10, wherein the method is a method for inducing from about 65% to 100% tetraploid production.

Paragraph 12. The method of any one of paragraphs 1-11, wherein a survival rate of larvae from the eggs that are exposed to the pressure shock is about 10% or greater compared to the survival rate of larvae from fish eggs that have not been exposed to the pressure shock.

Paragraph 13. The method of paragraph 12, wherein the survival rate is about 15% or greater.

Paragraph 14. The method of any one of paragraphs 1-13, wherein exposing the eggs to the pressure shock comprises exposing the eggs to the pressure for from about 1° C. minute to about 10° C. minutes.

Paragraph 15. The method of any one of paragraphs 1-14, wherein exposing the eggs to the pressure shock comprises exposing the eggs to the pressure for from about 5° C. minute to about 10° C. minutes.

Paragraph 16. A method of inducing from about 85% to 100% tetraploid production, comprising:
selecting fish eggs at between about 65% to about 75% FCI;
exposing the eggs to a pressure shock comprising a pressure from about 7,250 psi to about 8,250 psi for from about 3° C. minutes to about 7° C. minutes; and
allowing the eggs to hatch to produce larvae having from about 85% to 100% tetraploidy.

Paragraph 17. A method for producing a sterile population of fish, comprising:
introducing a population of tetraploid fish of a selected species to a population of diploid fish of the selected species as the tetraploid fish; and
allowing the tetraploid fish to breed with the diploid fish, thereby producing a population of sterile triploid fish of the selected species.

Paragraph 18. A method of reducing a population of an invasive species of fish in a water system, the method comprising introducing to the water system a plurality of tetraploid fish of the invasive species.

### VI. EXAMPLES

### Example 1

### Broodstock Management

All experiments were conducted using a captive burbot broodstock held in a recirculating aquaculture system. The broodstock population averaged 1.4 kg and 56.8 cm and 5 to 13 years of age. The Recirculating Aquaculture System (RAS) was managed to simulate the annual photoperiod and temperature conditions present in the Kootenai River in Bonners Ferry, Idaho. The broodstock were fed a sinking commercial diet, 6 mm pellets of Bio-brood and Oncor in a 1:1 ratio, at about 0.3% of tank biomass, dispensed from dusk until dawn via belt autofeeder. Fish were allowed to reach sexual maturity naturally, through exposure to low temperatures and the increase in daylight after the winter solstice. After the first occurrence of egg release in brood tanks the spawning condition of all fish was assessed three times each week throughout the spawning season.

### Example 2

### Gamete Collection and Fertilization

Gamete collection began by first anesthetizing ripe fish in buffered 150 ppm tricaine methanesulfonate (MS-222; Western Chemical, WA, USA) for three minutes. Anesthetized fish were then patted dry with a towel and gametes were stripped into a clean dry vessel upon an ice bath. After mixing, gametes were immediately activated with 2 L of 2° C. water, total egg to milt ratio was about 125:1 for all experiments. At 30- and 60-seconds post fertilization fresh milt was added to the combined gametes as this procedure has demonstrated enhanced fertilization success. At 2 minutes post fertilization, the milt/water mix was decanted, and eggs were resuspended in 2 L clean water at 2° C. Eggs were exposed to 25 ppm ovadine at 35 minutes post fertilization, for 15 minutes; after ovadine treatment, the water/ovadine mixture was decanted and eggs were resuspended in 2 L clean water at 2° C. Finally, at 90 minutes post fertilization, after water hardening had completed, eggs were placed in their respective incubator at 2° C.

For all experiments, each replicate was composed of one ripe female and two ripe males, selected at random. Females were considered ripe when eggs flowed freely from vent, males were considered ripe when they expressed milt with light stripping. Prior to fertilization, milt was assessed for adequate motility and eggs were inspected to ensure viability and any gametes determined to be of low quality were not used in these experiments. Females were spawned once and not used for subsequent replicates; males were used for multiple replicates due to the random selection process.

### Example 3

### Tetraploid Induction Experiments—Determination of First Cleavage Interval

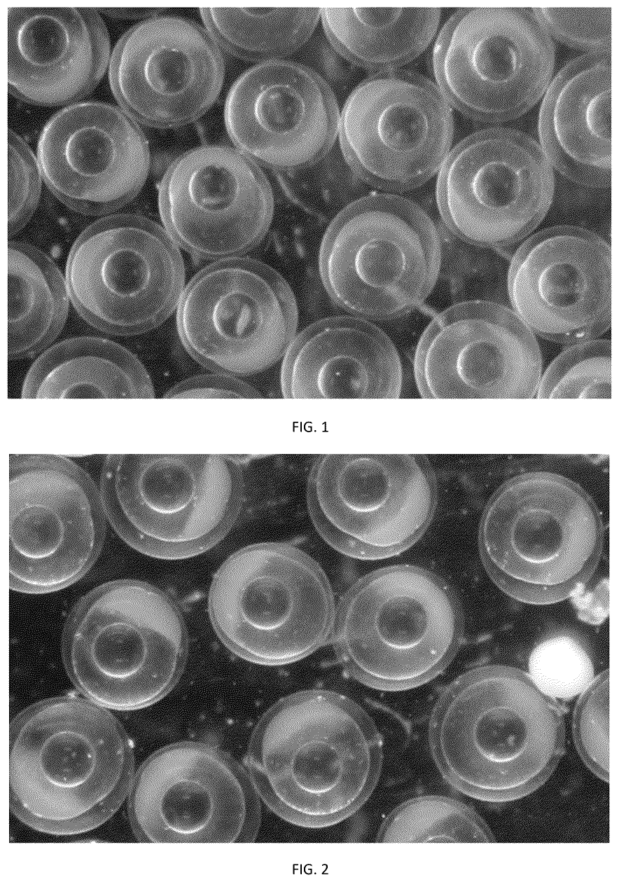

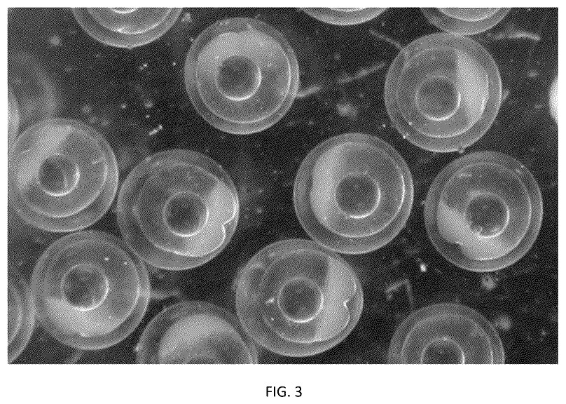

First cleavage interval (FCI) was determined for each replicate female by partially stripping a sample of eggs, approximately 10% of total spawn, then fertilizing and incubating as described previously. The remaining fraction of the spawn, approximately 90%, was retained within the respective female and recovered later for shock experiments. Starting 6 hours after fertilization of the sample, three subsamples of about 100 eggs were collected from the incubator every 30 minutes and photographed, sampled eggs were then returned to respective incubator. The FCI for each replicate female was defined as the first time in which ≥80% of the zygotes expressed the first cleavage (FIGS. 1-3).

Approximately 7 hours after fertilization of the sample eggs, the remaining spawn was collected from the respective female then fertilized and incubated using the methods described previously. The milt utilized for determination of FCI and fertilization of remaining fraction of the spawn originated from the same males and was freshly collected prior to utilization in all cases.

### Example 4

### Tetraploid Induction Experiments—Shock Treatments

The hydrostatic intensity and duration of shock used in this experiment was identical for all treatments, 7500 psi, for 10° C. minutes. Experimental units, about 2.5 mL, were collected from the respective incubator and placed in shock apparatus about 5 minutes prior to the shock application. Shocks were applied at 60, 65, 70, 75, 80, 85, 90, 95, or 100% FCJ. After the shock each experimental unit was volumetrically quantified via graduated cylinder and placed in a 10 mL incubation tube. Each incubation tube was then randomly placed in a mass incubator that incubated all experimental units in a shared water bath at 2° C. for the duration of incubation. The mass incubator and individual tubes were checked daily to remove dead eggs and ensure optimum performance. When approximately 20% of the experimental units began to hatch, the incubation temperature was increased to 8° C. to synchronize hatch to a one-week period. One day prior to hatch synchronization, all experimental units were volumetrically quantified via graduated cylinder after a final removal of dead eggs.

### Example 5

### Tetraploid Induction Experiments—Ploidy Analysis

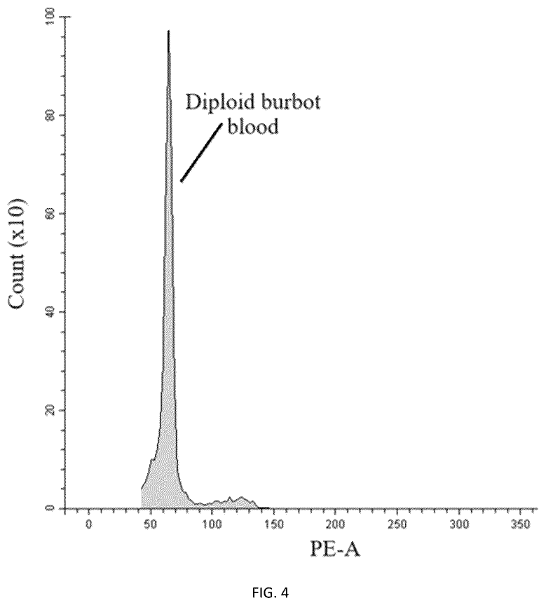

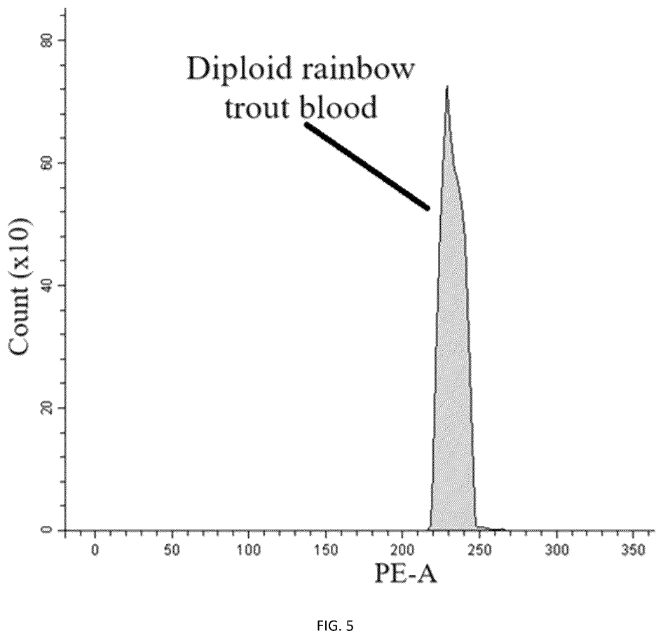

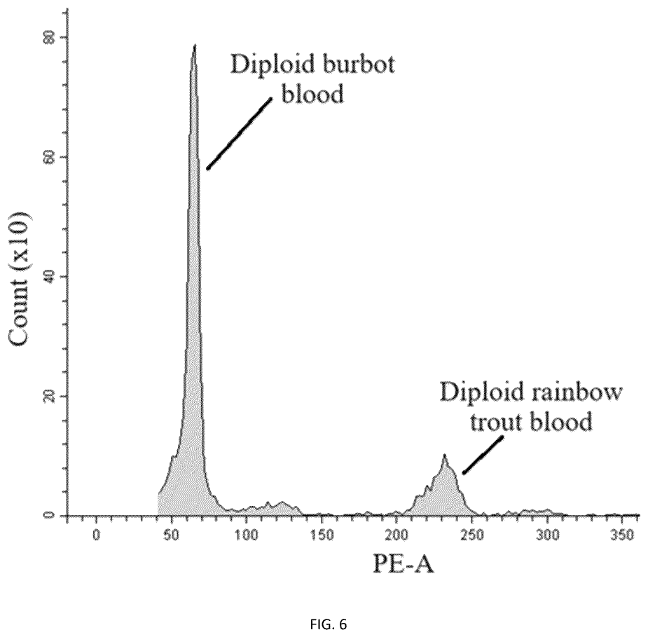

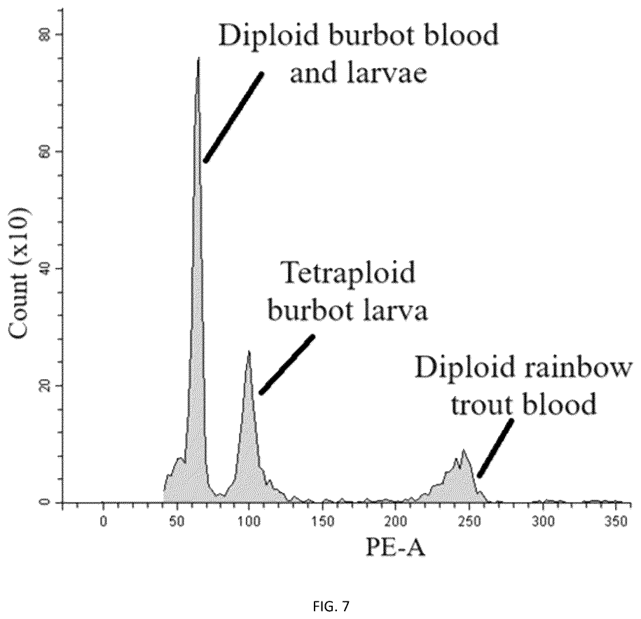

Larvae, 10 from each surviving experimental unit, were randomly sampled 3 days post hatch for nuclear DNA content via flow cytometry. DNA staining and ploidy analysis was carried out. Each sample consisted of a single larvae in 800 μL staining solution (50 mg L⁻¹ propidium iodide (VWR, PA, US), 10 mg L⁻¹ RNase A (VWR, PA, US) in Isoton II solution (VWR, PA, US)), two internal standards were also included in each sample (1 μl of a 0.05× dilution of diploid rainbow trout erythrocytes (Biosure Inc., CA, US) in Isoton II solution and 1 μl of a 0.05× dilution of diploid burbot blood (University of Idaho, ID, US) in Isoton II). Samples were incubated for 24 hours, vortexed, and then each larva was aspirated through a 26-gauge needle via a 1 mL syringe (VWR, PA, US) to separate the larvae into individual cells. During the last aspiration, samples were drawn into the syringe, the needle was replaced with a clean duplicate and 60 μm mesh (Ablazecustom, Hong Kong, CN) was placed between the syringe and needle; the sample was then filtered into a clean microcentrifuge tube. For each sample, 50,000 events were recorded using a Beckman Coulter Cytoflex S flow cytometer (Beckman Coulter Inc., CA, US). A tetraploid was confirmed when the mean fluorescence intensity of the sample peak was 2.0 times the mean fluorescence intensity of the internal standard peak (FIGS. 4-7).

### Results and Discussion from Examples 1-5

### Results

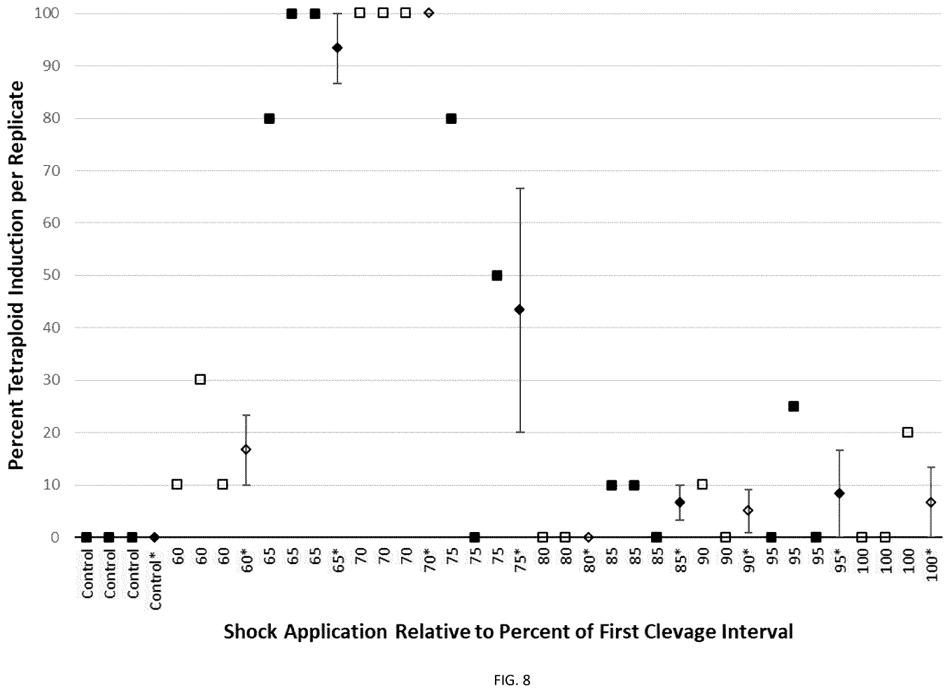

A pressure shock of 7,500 psi administered for 10° C. minutes at 70% FCI induced tetraploidy in 100% of the larvae sampled (Table 1 and FIG. 8).

TABLE 1
Tetraploid induction and associated survival.

| FCI when | Tetraploid | Relative |
|---|---|---|
| shocked (%) | induction (%) | survival (%) |
| No shock | 0.0^{c} | 100.0^{a} |
| 60 | 16.7^{bc} | 86.7^{a} |
| 65 | 93.3^{a} | 38.0^{a} |
| 70 | 100.0^{a} | 30.1^{a} |
| 75 | 65^{ab} | 14.2^{a} |
| 80 | 0^{c} | 0.7^{ab} |
| 85 | 6.7^{c} | 59.5^{a} |
| 90 | 5^{c} | 24.6^{a} |
| 95 | 8.3^{c} | 32.2^{a} |
| 100 | 6.7^{c} | 15.5^{b} |

Treatments utilizing a pressure shock at 75, 70, and 65% of FCI yielded the highest relative rates of tetraploid induction and were statistically similar to each other. Furthermore, shocks applied at 75% FCI produced tetraploid induction rates which were also statistically similar to those observed for shocks applied at 60% FCI. Tetraploid induction rates observed for shocks applied at 60% FCI were also statistically similar to those yielding the lowest rates of tetraploid induction, treatments shocked at 80 through 100% FCI as well as the controls which received no shock. Relative survival was statistically similar for all treatments except those shocked at 80% FCI, which were significantly lower relative to the controls but statistically similar to the rest of the treatments. Finally, examination of FCI occurrence for each female illustrated a slightly different time from fertilization to first cleavage (Table 2).

TABLE 2
Timing of first cleavage interval, °
C. hours:minutes post fertilization.

| Female | First cleavage interval (° C. hours: minutes) |
|---|---|
| A | 14:00 |
| B | 15:30 |
| C | 15:00 |

The egg storage experiments showed that when unfertilized burbot eggs were held, inactivated, at 4° C. up to 24 hours, percent survival was not significantly different from eggs that were immediately fertilized upon release from the female. Additionally, percent survival values for eggs stored up to 12 or 24 hours were not significantly different relative to eggs stored for 36 or 48 hours (Table 3).

TABLE 3
Egg storage survival

| Storage time (hours) | Relative survival (%) |
|---|---|
| 0 | 100.0^{a} |
| 12 | 36.7^{ab} |
| 24 | 25.3^{ab} |
| 36 | 0.5^{b} |
| 48 | 0.6^{b} |

### Discussion

This is the first experiment to identify parameters that result in 100% tetraploid induction within a burbot cohort. The results presented here describe 100% tetraploid induction in burbot, using a shock of 7,500 psi for 10° C. minutes at 70% FCI.

Through this research, it was observed that the timing of FCI in burbot varies by population and individual. The present experiment indicated a different FCI occurrence for each of the fish examined, with an average total FCI of 14 hours and 50 minutes (Table 2). Furthermore, ova aging has been shown to increase total FCI, and this should be accounted for and controlled to efficiently generate consistent maximum tetraploid yields. Thus, the variation in total FCI within the burbot population used herein may have resulted due to differences in ova aging; however, the natural total FCI variation may be within limits to allow the determination of a single shock point to effectively induce tetraploidy population wide.

Many studies have described the depressed growth and survival of first-generation tetraploid fish, relative to diploid controls, and noted the rarity of developing a tetraploid strain. First-generation referring to the fact that the tetraploids are derived from diploid parents. When sexually mature tetraploids are achieved and bred with diploids, the resulting autotriploid offspring often exhibit survival and growth that is comparable to their diploid counterparts and better than shock-induced triploids. Likewise, second generation tetraploids, created by breeding first generation tetraploids together, have been reported to exhibit 100% higher survival and reductions of abnormalities by 90%, relative to first generation tetraploids. The prominent issues preventing the establishment of tetraploid broodstock in finfish are the low survival of first generation tetraploids, reduced fertility of mature tetraploids, and unfavorable ploidy in tetraploid offspring. Generation of mature tetraploid burbot has a reasonable chance of success due to the high fecundity of the species.

### Example 6

### Introduction

In burbot, a hydrostatic shock of 7,500 psi for 5° C. minutes at 70% of the first cleavage interval (FCI) resulted in 100% tetraploid induction with survival statistically similar (p≥0.05) to unshocked control eggs. The units ° C. minutes is simply the duration of exposure to the egg temperature at shocking. Tetraploid state was initially confirmed with flow cytometry; however, a secondary methodology of ploidy confirmation is necessary.

### Methods

### Tetraploid Induction and Rearing

Two burbot females are partially spawned and eggs fertilized to determine the FCI for the respective eggs. The remaining eggs, unfertilized and within the female are collected and fertilized, then shocked at the now known 70% FCI. The Eggs used to determine the FCI are reared as negative controls.

Eggs are incubated and larvae reared using standard procedures utilized at the Aquatic Animal Research Facility at the University of Idaho.

### Determination of Tetraploid State

Juvenile burbot, 10 family⁻¹, from the tetraploid induction and negative control treatments are anesthetized with buffered 80 ppm tricaine methanesulfonate. After apparent anesthesia blood is collected from each fish. The blood is used to assess ploidy via flow cytometry and the remaining burbot tissue is sent away for karyotype analysis.

### CONCLUSION

This analysis will confirm the results of the flow cytometric analysis of ploidy, and provide a certainty of tetraploid status.

In view of the many possible aspects to which the principles of the disclosure may be applied, it should be recognized that the illustrated aspects are only preferred examples of the disclosure and should not be taken as limiting the scope of the disclosure. Rather, the scope of the disclosure is defined by the following claims. We therefore claim as the disclosure all that comes within the scope and spirit of these claims.

We claim:

1. A method, comprising:
selecting fish eggs at between about 65% to about 75% FCI;
exposing the eggs to a pressure shock comprising a pressure from about 7000 psi to about 9000 psi for from about 1° C. minutes to about 20° C. minutes.

2. The method of claim 1, wherein the method is a method of inducing from about 50% to 100% tetraploid production.

3. The method of claim 1 wherein the method is a method of inducing from about 60% to 100% tetraploid production.

4. The method of claim 1 wherein the fish eggs are from a cold-water species of fish.

5. The method of claim 4, wherein the cold-water species of fish is a freshwater species.

6. The method of claim 1, wherein the fish eggs have an average size of from greater than zero to about 5 mm.

7. The method of claim 6, wherein the average size of the fish eggs is from about 0.5 mm to about 3 mm.

8. The method of claim 6, wherein the average size of the fish eggs is from about 0.7 mm to about 2 mm.

9. The method of claim 1, wherein the fish eggs are eggs from fish selected from carp, walleye, yellow perch, burbot, Yellowtail Kingfish and amberjack species (genus Seriola), sablefish (black cod), red drum, cobia, flounders, Atlantic cod, pacific cod, sea bass species, mahi mahi, tilapia, large mouth bass, or catfish.

10. The method of claim 9, wherein the fish eggs are burbot eggs.

11. The method of claim 1, wherein the pressure is from about 7,250 psi to about 8,500 psi.

12. The method of claim 1, wherein the method is a method for inducing from about 65% to 100% tetraploid production.

13. The method of claim 1, wherein a survival rate of larvae from the eggs that are exposed to the pressure shock is about 10% or greater compared to the survival rate of larvae from fish eggs that have not been exposed to the pressure shock.

14. The method of claim 13, wherein the survival rate is about 15% or greater.

15. The method of claim 1, wherein exposing the eggs to the pressure shock comprises exposing the eggs to the pressure for from about 1° C. minute to about 10° C. minutes.

16. The method of claim 1, wherein exposing the eggs to the pressure shock comprises exposing the eggs to the pressure for from about 5° C. minute to about 10° C. minutes.

17. The method of claim 1, comprising:
selecting fish eggs at between about 65% to about 75% FCI;
exposing the eggs to a pressure shock comprising a pressure from about 7,250 psi to about 8,250 psi for from about 3° C. minutes to about 7° C. minutes; and
allowing the eggs to hatch to produce larvae having from about 85% to 100% tetraploidy.

18. The method of claim 17, wherein the fish eggs are burbot eggs.

19. A method for producing a sterile population of fish, comprising:
introducing a population of tetraploid fish of a selected species to a population of diploid fish of the selected species as the tetraploid fish; and
allowing the tetraploid fish to breed with the diploid fish, thereby producing a population of sterile triploid fish of the selected species.

20. A method of reducing a population of an invasive species of fish in a water system, the method comprising introducing to the water system a plurality of tetraploid fish of the invasive species.
